(19) United States
(12) Patent Application Publication
(10) Pub. No.: US 2016/0262779 A1
(43) Pub. Date: Sep. 15, 2016

(54) Electro-Surgical Forceps Having Clad Metal Structure and Process for Manufacturing Same

(71) Applicant: Kirwan Surgical Products LLC, Mansfield, MA (US)

(72) Inventor: Lawrence T. Kirwan, JR., Kingston, MA (US)

(21) Appl. No.: 14/657,369

(22) Filed: Mar. 13, 2015

(51) Int. Cl. A61B 17/29

(52) U.S. Cl. CPC A61B 17/29; A61B 2017/2948; A61B 2017/00526

(57) ABSTRACT

Electrosurgical forceps having a clad metal structure and a method of manufacturing electrosurgical forceps using a cladding process are provided. The clad metal structure of the forceps includes a first layer of non-stick material, which may be copper, a copper alloy, silver, or a silver alloy, and a second layer of a material providing good mechanical properties and light weight, which may be aluminum, an aluminum alloy, titanium, a titanium alloy, or stainless steel.

### CROSS-REFERENCE TO RELATED APPLICATIONS

N/A

### STATEMENT REGARDING FEDERALLY SPONSORED RESEARCH OR DEVELOPMENT

N/A

### BACKGROUND

Electro-surgical forceps have a pair of resilient blades or arms that are used for grasping and coagulating tissue. The forceps may be monopolar or bipolar. In monopolar forceps, the blades are welded or otherwise joined to form an electrode in electrical communication with an electro-surgical generator. Current flows from the active electrode through the patient's tissue to a dispersive electrode in contact with the patient's skin (which may be at some distance from the forceps) and back to the generator. In bipolar forceps, each blade of the pair comprises an electrode in communication with an electro-surgical generator. Current flows from one blade through the tissue to the other blade.

In some instances, tissue may adhere or stick to the tips of the blades. If sticking occurs, the surgeon must pull on the forceps to release it from the tissue, possibly causing further bleeding and requiring that the forceps be cleaned. It is known to prevent or minimize such sticking of tissue to electrosurgical forceps by manufacturing the blades of the forceps from nickel. See, for example, U.S. Pat. No. 5,196,009. During high power operation, some eschar buildup and some sticking of the tissue to the tips still may occur.

Another known manner of preventing or minimizing sticking is to form the blades from a metal or metal alloy having a relatively high thermal conductivity, such as copper, that is able to transfer heat away from the tips of the blades. By keeping the tissue cooler, for example, below the boiling point of water, coagulation is able to occur without sticking of the tissue. See, for example, U.S. Pat. No. 4,492,231. Nickel is more biocompatible with human tissue than copper and is preferable for contact with tissue, as well as proving additional non-stick capabilities. Thus another known forceps provides blades formed of an inner layer of copper or copper alloy having a thickness sufficient to dissipate heat and an outer covering of a strong, biocompatible metal or metal alloy such as nickel. See U.S. Pat. Nos. 6,059,783 and 6,298,550, the disclosures of which are incorporated by reference herein.

U.S. Pat. No. 6,749,610, the disclosure of which is incorporated by reference herein, discloses an electro-surgical forceps in which at least one of the tines has an outer plating that covers all or substantially all of the tine. The outer plating includes silver, rhodium, gold, aluminum, palladium, tungsten, or nickel.

### SUMMARY OF THE INVENTION

Electrosurgical forceps having a clad metal structure and a method of manufacturing electrosurgical forceps using a cladding process are provided. In the cladding process, two metal or metal alloy strips or sheets are rolled together under high pressure, resulting in a metallurgical bond between the two materials in each layer.

In one embodiment, an electro-surgical forceps comprises an insulated cap portion; at least one terminal extending from and fixed to the cap portion; and a pair of blade members, each blade member being generally elongated and having a tip and an opposite end fixed within the cap portion. At least one of the pair of blade members is electrically connected to the at least one terminal within the cap portion and comprises a clad metal structure. The clad metal structure comprises a first layer of a first material having a thickness sufficient to dissipate heat generated at the tip to prevent sticking of tissue to the forceps during use, the first material comprising at least one of copper, a copper alloy, silver, or a silver alloy. The clad metal structure further comprises a second layer of a second material having a thickness sufficient to provide greater mechanical strength than the first layer, the second material comprising at least one of aluminum, an aluminum alloy, titanium, a titanium alloy, or stainless steel. The second layer is bonded with a metallurgical bond to the first layer.

In another aspect of the forceps, the thickness of the first layer ranges from 0.0001 to 0.020 inch. In another aspect of the forceps, the thickness of the second layer ranges from 0.050 to 0.120 inch. In another aspect of the forceps, a combined thickness of the first layer and the second layer ranges from 0.070 to 0.130 inch.

In another aspect of the forceps, the silver alloy of the first layer comprises at least 80% silver. In another aspect of the forceps, the copper alloy of the first layer comprises at least 80% copper. In another aspect of the forceps, the aluminum alloy of the second layer comprises at least 80% aluminum. In another aspect of the forceps, the titanium alloy of the second layer comprises at least 80% titanium.

In another aspect of the forceps, the second layer is metallurgically bonded to the first layer sufficiently to prevent delamination from the first layer.

In another aspect of the forceps, a plating of an electrically and thermally conductive biocompatible material is provided over at least a tip end of the blade members. In another aspect of the forceps, the plating comprises gold.

In another aspect of the forceps, an insulating coating is provided over the first layer and the second layer, and extending from the cap portion to a location near the tip.

In a further embodiment, a process of manufacturing an electro-surgical forceps comprises:
providing a strip comprising a clad metal layered structure comprising a first layer of a first material and a second layer of a second material, the first material comprising at least one of copper, a copper alloy, silver, or a silver alloy, and the second material comprising at least one of aluminum, an aluminum alloy, titanium, a titanium alloy, or stainless steel;
cutting the strip into a first blade member having a blade configuration extending from a proximal end to a distal end, a tip disposed at the distal end;
providing a second blade member;
connecting the first blade member and the second blade member to electrodes at a connection; and
fixing the connection between the blade member, the second blade member and the electrodes within an insulating cap portion.

In another aspect of the process, the step of providing the strip comprising the clad metal layered structure comprises cladding the first layer to the second layer under pressure.

In another aspect of the process, the cladding step comprises feeding the first layer and the second layer into a rolling mill.

In another aspect of the process, the step of cutting the strip into the first blade member comprises water jet cutting with a water jet at a pressure of up to 100,000 psi. In another aspect of the process, an abrasive material is entrained into the water jet.

In another aspect of the process, the step of cutting the strip into the first blade member comprises water jet cutting, blanking, laser cutting, or plasma cutting.

In another aspect of the process, an electrically conductive material is plated on at least a tip of the blade member.

In another aspect of the process, portion of the blade member is encapsulated in an insulating material.

### DESCRIPTION OF THE DRAWINGS

The invention will be more fully understood from the following detailed description taken in conjunction with the accompanying drawings in which:

FIG. 1 is a side view of an embodiment of electro-surgical forceps;

FIG. 2 is a partially sectional, plan view of the forceps of FIG. 1;

FIG. 3 is a cross-sectional view along line III-III of FIG. 2;

FIG. 4 is a cross-sectional view of a further embodiment;

FIG. 5 is a cross-sectional view of a still further embodiment;

FIG. 6 is a schematic view of a cladding process for manufacture of electro-surgical forceps; and

FIG. 7 is a plan view of clad metal strip stock;

FIG. 8A is a plan view of a blade member cut from the strip stock of FIG. 7;

FIG. 8B is a side view of the blade member of FIG. 8A;

FIG. 9A is a plan view of the blade member of FIG. 8A after further forming; and

FIG. 9B is a side view of the blade member of FIG. 9A.

### DETAILED DESCRIPTION OF THE INVENTION

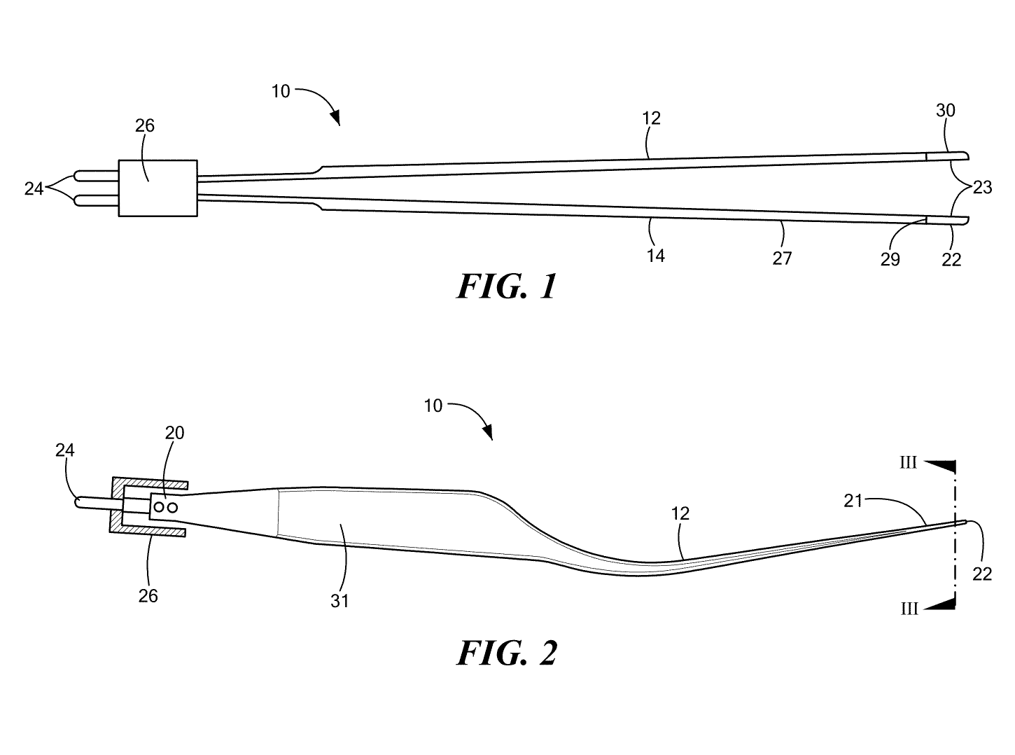

Referring to FIGS. 1 and 2, an electro-surgical forceps has first and second blade or electrode members 12, 14. Each of the blade members are elongated and extend from a first or proximal end 20 to a second or distal end 21 having a tip 22. The blades are generally flat to have a greater width than depth, such that the tips are configured for gripping tissue between opposed surfaces 23. First ends 20 are electrically connected in any suitable manner, such as by crimping, welding, or soldering, to terminal pins 24. First ends 20 along with the terminal pins 24 are encapsulated using an epoxy-based material or otherwise mounted within an insulating cap portion 26. The blades are insulated with an insulating material 27 along most of their length from the cap portion 26 to a location 29 close to the tip. Finger grips, for example, serrations (not shown), may be formed on each blade member at a suitable gripping location 31 to aid the physician in gripping the forceps during use. A plating of an electrically and thermally conductive biocompatible material such as gold may be provided on the tip 22 or extending along a portion of the length or the entire length of each blade. Bipolar forceps are shown; however, the forceps could be monopolar forceps as well.

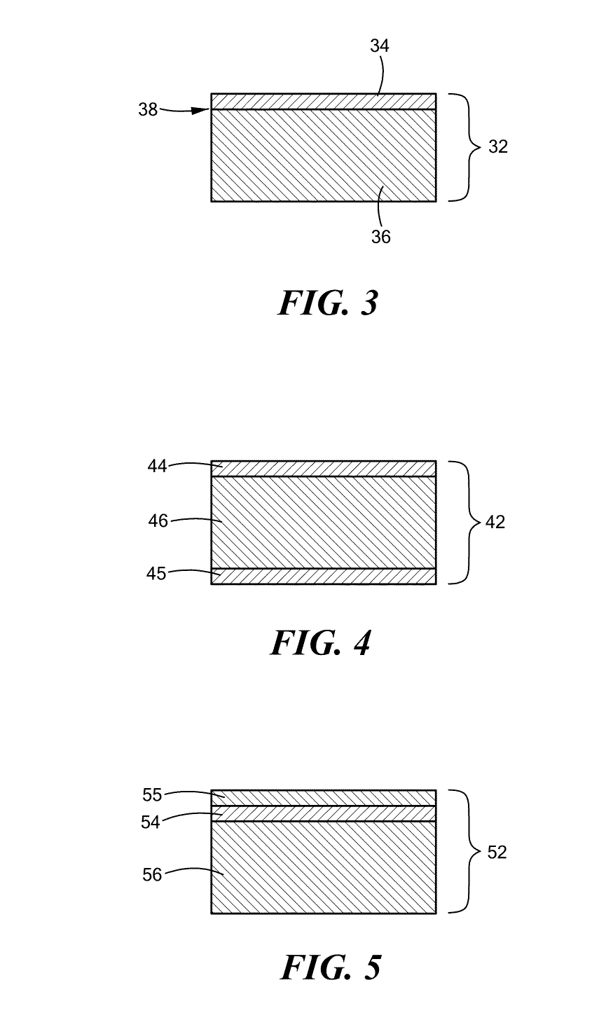

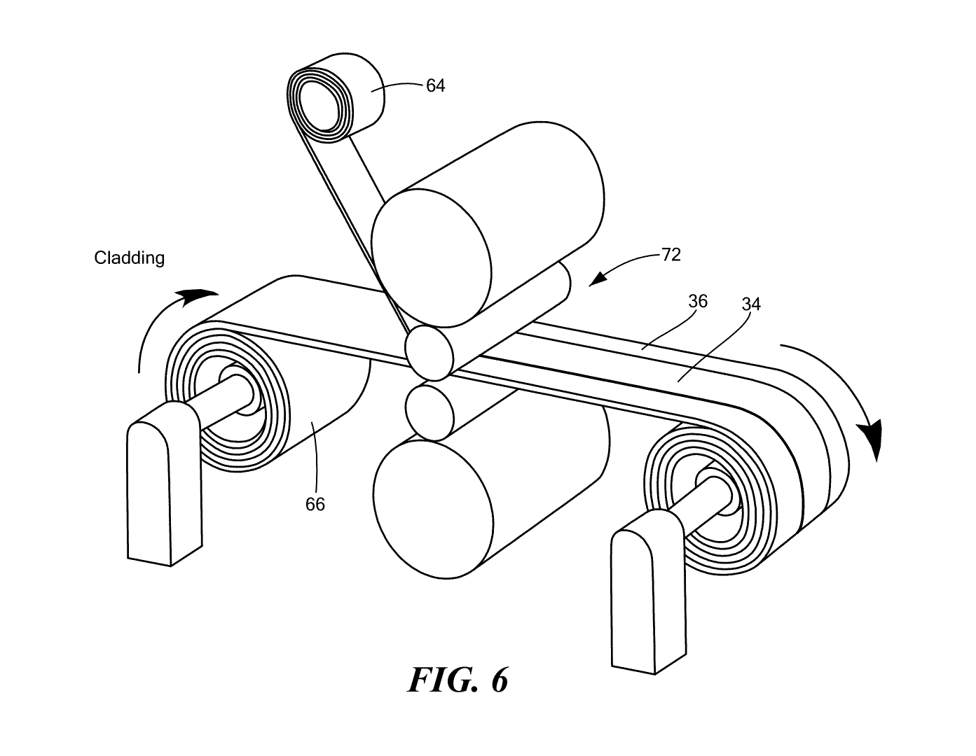

Referring more particularly to FIG. 3, at least one and, in some embodiments both, of the blade members 12, 14 comprise a clad metal bi-layered planar structure 32 having a first layer 34 of a first material bonded with a metallurgical bond 38 (illustrated schematically) through a cladding process to a second layer 36 of a second material. The first layer 34 provides good thermal conductivity to dissipate heat from the tip of the forceps sufficient to prevent sticking of tissue to the forceps during use. The second layer 36 provides both good mechanical strength to support the first layer and light weight. Additional layers could be used if desired. For example, a tri-layered structure 42 could be provided, in which a layer 46 of the second material is sandwiched between layers 44, 45 of the first material. See FIG. 4. FIG. 5 illustrates a further embodiment of a tri-layered structure 52, in which a first layer 54 of a first material is bonded between a layer 55 of a third material and a layer 56 of a second material. Other layered structures, having different material configurations and different number of layers, including more than three layers, can be used if desired.

Referring again to FIG. 3, the first material of the first layer 34 can be at least one of copper, a copper alloy, silver, or a silver alloy. The second material of the second layer 36 can be at least one of aluminum, an aluminum alloy, titanium, a titanium alloy, or stainless steel. Referring to FIG. 5, the first material of the layer 54 can be copper or a copper alloy, the third material of the layer 55, can be at least one of silver or nickel or alloys thereof or other biocompatible material, and the second material of the layer 56 can be at least one of aluminum, an aluminum alloy, titanium, a titanium alloy, or stainless steel.

If an alloy is used, the base material (silver, copper, aluminum, titanium) is typically at least 80% of the alloy. For example, an alloy of silver could be at least 80% silver, at least 85% silver, at least 90% silver, or at least 95% silver. An alloy of copper could be at least 80% copper, at least 85% copper, at least 90% copper, or at least 95% copper. An alloy of aluminum could be at least 80% aluminum, at least 85% aluminum, at least 90% aluminum, or at least 95% aluminum. An alloy of titanium could be at least 80% titanium, at least 85% titanium, at least 90% titanium, or at least 95% titanium. Any suitable stainless steel can be used. One suitable alloy is aluminum 6061 T6.

The thickness of the first layer 34 (also layers 44, 45, 54, 55) can suitably range from 0.0001 to 0.020 inch. In some embodiments, the thickness of the first layer can be 0.0025 inch, 0.0005 inch, 0.0075 inch, 0.001 inch, 0.025 inch, 0.004 inch, 0.005 inch, 0.006 inch, 0.075 inch, 0.010 inch, or 0.015 inch. The thickness of the second layer 36 (also layers 46, 56) can suitably range from 0.050 to 0.120 inch. In some embodiments, the thickness of the second layer can be 0.060 inch, 0.070 inch, 0.075 inch, 0.080 inch, 0.090 inch, 0.10 inch, or 0.11 inch. The total thickness of both layers in the layered structure 32 (or structures 42, 52) can range from 0.0501 to 0.140 inch, or in some embodiments, from 0.070 to 0.130 inch. Other layer thicknesses can be used if desired, depending on the application.

As mentioned above, the first and second layers 34, 36 are bonded together with a cladding process to produce the clad metal structure. Referring to FIG. 6, the first layer 34 and the second layer 36 are preferably supplied as separate components 64, 66 as sheet or strip stock. Each component is cleaned to remove dirt, oxides and other impurities that may be present. The components 64, 66 are fed into a rolling mill 72 and cold bonded under high pressure, reducing the thickness to desired thicknesses of each layer and bonding the components together. The pressure typically ranges up to 8 million psi. The starting thickness of each component and the pressure applied in the mill can be selected to arrive at the desired final thicknesses. A metallurgical bond is created due to the high pressure applied in the rolling mill. The material can then be annealed to establish a specific heat treatment. For example, the T6 temper for aluminum involves holding the material for one hour at 400° F. and eight hours at 325° F. The particular heat treatment applied depends on the material and application. Although referred to as “cold” bonding, the temperature of the material exiting the mill is typically greater than 300 to 400° F. The temperatures of hot bonding techniques, however, are typically greater than 1000° F. A hot bonding technique could be used if desired, for example, to improve cladding adhesion. A similar process can be used when more than two layers are bonded together.

As noted above, the cladding process produces a metallurgical bond between the two layers. With a metallurgical bond, the material fractures before the layers separate. A metallurgical bond is formed at the molecular and atomic level, such that the lattice structures of the adjacent metals or metal alloys are forced into conformity with an accompanying combining of molecules between the two materials. A cladding process is useful for combining metals or metal alloys having different properties or characteristics, such as electrical properties, magnetic properties, mechanical properties, thermal properties, corrosion resistance, biocompatibility, and cost. The amount and placement of each material can be controlled to achieve a desired combination of properties in the finished product.

The cladding process provides several advantages over a plating process. The final composition of the finished product is more consistent. Some materials cannot be plated or do not lend themselves to a plating process and thus cannot be combined with another material in a plating process. The cladding process can produce a denser, harder, more wear resistant surface than can be produced by a plating process. The bond between the two materials is a metallurgical bond, in which the metals share molecules due to the forced conformance of their lattice structures, and thus is stronger than the bond resulting from a plating process.

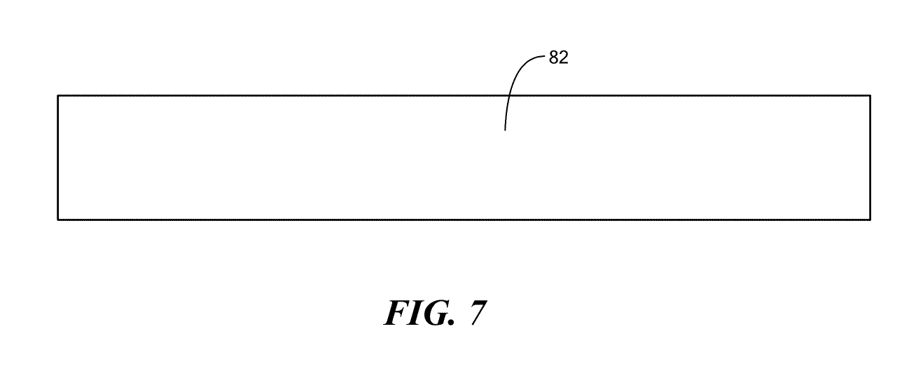

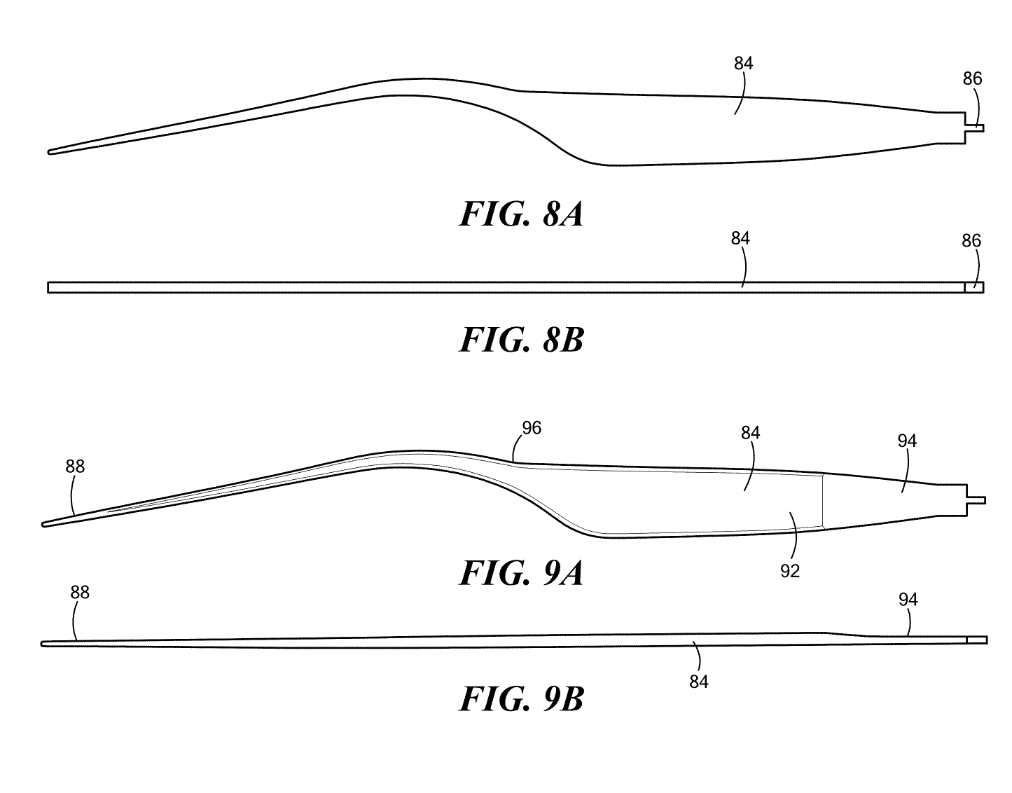

FIGS. 7-9B illustrate representative steps in the process of manufacturing a blade member with a clad metal structure. FIG. 7 illustrates a clad metal strip stock 82 after the cladding process described above with reference to FIG. 6. The strip 82 is cut to a desired configuration for a blade member 84. See FIG. 8A. The blade member can have any suitable configuration, such as a curved configuration as shown in FIGS. 2, 8A, and 9A. Other curved configurations or a straight configuration can be provided, if desired. A tab 86 can be cut at the proximal end for subsequent connection to an electrical terminal.

In one embodiment, a water jet cutting process is used to cut the strip 82 for the blade member 84. A water jet cutting process prevents the softer metal or metal alloy of the first layer (copper, silver, and their alloys) from squeezing out along the perimeter, which typically occurs in stamping processes used in prior art manufacturing processes. The water jet can be provided at high pressures of, for example, up to 100,000 psi. In some embodiments, a higher pressure can be used. An abrasive material, such as particles of aluminum oxide or garnet, can be entrained into the water jet to assist in the cutting process. In other embodiments, another cutting process, such as blanking, laser cutting or plasma cutting, can be used.

A taper 88 can be stamped at the distal end to the tip of the blade member. If desired, serrations or another gripping structure (not shown) can be stamped or otherwise provided at a gripping location 92. A rear or spring section 94 can be cold formed as by stamping or coining, to compress its thickness and to work harden the material. Work hardening strengthens the material, enabling a physician to squeeze the blades together repeatedly to grasp tissue and release the blades to return to their rest position. The perimeter 96 of the blade member can be formed, for example, by a coining process or by milling and deburring, to form the edges. Progressive dies can be provided to perform some or all of these steps.

The blade, or any desired portion of the blade, such as the tip, can also be plated with a further material if desired. For example, if the blade includes copper or a copper alloy, at least the tip is preferably plated with a thin layer of a biocompatible material, such as gold, using conventional plating processes. A gold plating can also be applied for aesthetic purposes, for example, on a blade that includes silver or a silver alloy. The thickness of the plating generally ranges from 0.0001 to 0.001 inches. Any desired length of the blade can be plated. For example, in one embodiment, only the tip is plated; in another embodiment, the entire length of the blade is plated; in a further embodiment, an intermediate length of the blade is plated. Any suitable plating process can be used.

The blade member can be encapsulated in an insulating material, such as a plastic material capable of withstanding the high temperatures generated during use. The insulation can be formed in any suitable manner, such as by spraying on a liquid that dries to form a solid coating. The tip of the blade member is left uninsulated for a suitable distance, such as ⅜ inch. The insulation is typically 0.005 to 0.015 inch thick.

The forceps are assembled from two blade members having corresponding or complementary configurations. The terminal pins may be attached to the tabs of each blade member in any suitable manner, such as by crimping, welding, or soldering. Holes may be cut or stamped into the end (see FIG. 2) to allow epoxy or other appropriate potting material to flow through and around the blades to fix the blades more firmly within the cap portion.

It will be appreciated that the various features of the embodiments described herein can be combined in a variety of ways. For example, a feature described in conjunction with one embodiment may be included in another embodiment even if not explicitly described in conjunction with that embodiment.

The present invention has been described with reference to the preferred embodiments. It is to be understood that the invention is not limited to the exact details of construction, operation, exact materials or embodiments shown and described, as obvious modifications and equivalents will be apparent to one skilled in the art. It is believed that many modifications and alterations to the embodiments disclosed will readily suggest themselves to those skilled in the art upon reading and understanding the detailed description of the invention. It is intended to include all such modifications and alterations insofar as they come within the scope of the present invention.

What is claimed is:

1. An electro-surgical forceps comprising:
an insulated cap portion;
at least one terminal extending from and fixed to the cap portion; and
a pair of blade members, each blade member being generally elongated and having a tip and an opposite end fixed within the cap portion;
at least one of the pair of blade members electrically connected to the at least one terminal within the cap portion and comprising a clad metal structure, the clad metal structure comprising:
a first layer of a first material having a thickness sufficient to dissipate heat generated at the tip to prevent sticking of tissue to the forceps during use, the first material comprising at least one of copper, a copper alloy, silver, or a silver alloy, and
a second layer of a second material having a thickness sufficient to provide greater mechanical strength than the first layer, the second material comprising at least one of aluminum, an aluminum alloy, titanium, a titanium alloy, or stainless steel,
wherein the second layer is bonded with a metallurgical bond to the first layer.

2. The forceps of claim 1, wherein the thickness of the first layer ranges from 0.0001 to 0.020 inch.

3. The forceps of claim 1, wherein the thickness of the second layer ranges from 0.050 to 0.120 inch.

4. The forceps of claim 1, wherein a combined thickness of the first layer and the second layer ranges from 0.070 to 0.130 inch.

5. The forceps of claim 1, wherein the silver alloy of the first layer comprises at least 80% silver.

6. The forceps of claim 1, wherein the copper alloy of the first layer comprises at least 80% copper.

7. The forceps of claim 1, wherein the aluminum alloy of the second layer comprises at least 80% aluminum.

8. The forceps of claim 1, wherein the titanium alloy of the second layer comprises at least 80% titanium.

9. The forceps of claim 1, wherein the second layer is metallurgically bonded to the first layer sufficiently to prevent delamination from the first layer.

10. The forceps of claim 1, further comprising a plating of an electrically and thermally conductive biocompatible material over at least a tip end of the blade members.

11. The forceps of claim 10, wherein the plating comprises gold.

12. The forceps of claim 1, further comprising an insulating coating over the first layer and the second layer, and extending from the cap portion to a location near the tip.

13. A process of manufacturing an electro-surgical forceps comprising:
providing a strip comprising a clad metal layered structure comprising a first layer of a first material and a second layer of a second material, the first material comprising at least one of copper, a copper alloy, silver, or a silver alloy, and the second material comprising at least one of aluminum, an aluminum alloy, titanium, a titanium alloy, or stainless steel;
cutting the strip into a first blade member having a blade configuration extending from a proximal end to a distal end, a tip disposed at the distal end;
providing a second blade member;
connecting the first blade member and the second blade member to electrodes at a connection; and
fixing the connection between the blade member, the second blade member and the electrodes within an insulating cap portion.

14. The process of claim 13, wherein the step of providing the strip comprising the clad metal layered structure comprises cladding the first layer to the second layer under pressure.

15. The process of claim 14, wherein the cladding step comprises feeding the first layer and the second layer into a rolling mill.

16. The process of claim 13, wherein the step of cutting the strip into the first blade member comprises water jet cutting with a water jet at a pressure of up to 100,000 psi.

17. The process of claim 16, further comprising entraining an abrasive material in to the water jet.

18. The process of claim 13, wherein the step of cutting the strip into the first blade member comprises water jet cutting, blanking, laser cutting, or plasma cutting.

19. The process of claim 13, further comprising plating an electrically conductive material on at least a tip of the blade member.

20. The process of claim 13, further comprising encapsulating a portion of the blade member in an insulating material.
